(19) United States
(12) Patent Application Publication
(10) Pub. No.: US 2024/0358324 A1
(43) Pub. Date: Oct. 31, 2024

(54) UROLOGICAL HEALTH DIAGNOSTIC

(71) Applicant: ConvaTec Technologies Inc., Las Vegas, NV (US)

(72) Inventors: Eric Luellen, Interlochen, MI (US); Diana Durieux, Copenhagen (DK); Reza Ghazi, Burlington, MA (US); Ased Ali, Wakefield (GB); Jacob Agris, New York, NY (US); Brian Murphy, Winchester, MA (US)

(21) Appl. No.: 18/769,322

(22) Filed: Jul. 10, 2024

Related U.S. Application Data
(63) Continuation of application No. PCT/US23/10589, filed on Jan. 11, 2023.
(60) Provisional application No. 63/298,961, filed on Jan. 12, 2022.

(51) Int. Cl. A61B 5/00; A61B 5/0205; A61B 5/024; A61B 5/20; G16H 50/20

(52) U.S. Cl. CPC A61B 5/486; A61B 5/0205; A61B 5/208; G16H 50/20; A61B 5/02438

(57) ABSTRACT

A method for identifying urological health information. The method includes storing user-defined inputs provided by a user, monitoring a fluid volume of urine processed by the user, storing parameters regarding the fluid volume of urine, utilizing a machine learning algorithm to provide processed data based on the user-defined inputs and stored parameters, and providing feedback based on the processed data.

### CROSS-REFERENCE TO RELATED DISCLOSURE

The present disclosure is a continuation of International Application No. PCT/US23/10589 filed Jan. 11, 2023 and claims the benefit of U.S. Provisional Application No. 63/298,961 filed on Jan. 12, 2022, the contents of which being incorporated herein in entirety.

### FIELD OF THE DISCLOSURE

The present invention relates to identifying urological health information and more specifically to identifying urological health information using one or more of user inputs, audio data, and machine learning.

### BACKGROUND OF THE DISCLOSURE

The present disclosure relates to monitoring urological information to identify a medical condition. Consumers who use medical devices at home, such as urological catheters, often misuse the devices or use them intermittently in contraindication to their designs or prescribed uses. Moreover, for medical device manufacturers, there is often no method by which to learn real-time feedback-including anxiety level and physiological data—to optimize device use, other than via simulation.

In one typical scenario, self-catheterization patients may decrease use because of anxiety or discomfort. Catheter regiment adherence, and proper bladder emptying, is of clinical relevance to prevent adverse events such as infections and autonomic dysreflexia, along with the associated costs.

Accordingly, there is a need for a system and method for easily monitoring and analyzing data from a user to identify and predict scenarios that may lead to an adverse event.

### SUMMARY

One aspect of this disclosure utilizes applications on smartphones, smartwatches, and other smart devices enable the real-time collection of physiological data associated with uses of medical devices to optimize adherence, proper use, clinical outcomes, and health economics.

One embodiment includes a smartwatch and smartphone application that record real-time one or more of fluids intake, anxiety levels, times, frequencies, and circumstances of catheterization, physiological data (e.g., blood oxygen saturation, blood pressure, heart-rate variability, temperature, heart rate), user feedback, a sound recording of urination to infer volume and post-void residual via machine learning, and Bluetooth monitors and sensors to capture, analyze, and proactively or responsively advise optimized catheter uses (e.g., frequency, duration, product selection, etc.).

One embodiment of this disclosure is a method for identifying urological health information. The method includes storing user-defined inputs provided by a user, monitoring a fluid volume of urine processed by the user, storing parameters regarding the fluid volume of urine, utilizing a machine learning algorithm to provide processed data based on the user-defined inputs and stored parameters, and providing feedback based on the processed data.

The monitoring step can include monitoring the volume of urine processed by a user through a urinary catheter. This method can use a microphone to record audio during a catheterization process to determine the fluid volume of urine transferred during the catheterization process with the machine learning algorithm. The method considered herein can also include storing details about the urinary catheter and considering the details to determine the fluid volume of urine transferred during the catheterization process. In one example, the details include a urinary catheter gauge. In another aspect of this example, the details can include the gender for which the urinary catheter is intended to be used. In examples considered herein, the microphone can be on a wristwatch, or smartwatch, or on a phone.

In other examples considered herein, the user-defined inputs can include a survey identifying anxiety. The user-defined inputs can also include fluid intake volume, activity level, lifestyle, diet, and other data that has dependency to insensible fluid loss. Part of this example can include determining a post-void residual volume with the machine learning algorithm based on the fluid intake volume, the fluid volume of urine transferred during the catheterization process, and an estimation of the insensible fluid loss

In another example, the user-defined inputs include one or more of a device type, gender, age, weight, height, specific injury, frequency of device use, and survey data.

Another example of this embodiment includes gathering and storing heart rate data and considering the heart rate data with the machine learning algorithm before providing feedback. This may be used to provide early prediction of autonomic dysreflexia among other thing.

In yet another example of this embodiment the feedback includes one or more of a recommendation for device use frequency, a recommendation for device type, and/or a medical recommendation.

Yet another example of this disclosure includes identifying and considering one or more of blood oxygen saturation, blood pressure, heart-rate variability, body temperature, and heart rate with the machine learning algorithm. This can be used to predict hydration levels and also early indications of urinary tract infection or autonomic dysreflexia among other things.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1a is a schematic flow chart of components of one embodiment of the present disclosure;

FIG. 1b is a schematic flow chart of one embodiment of a urological health diagnostic system implemented by the components of FIG. 1a;

FIG. 2 is a functional data flowchart of another embodiment of this disclosure; and

FIG. 3 is a data process flowchart of another embodiment of this disclosure.

Corresponding reference numerals indicate corresponding parts throughout the several views.

### DETAILED DESCRIPTION

The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure.

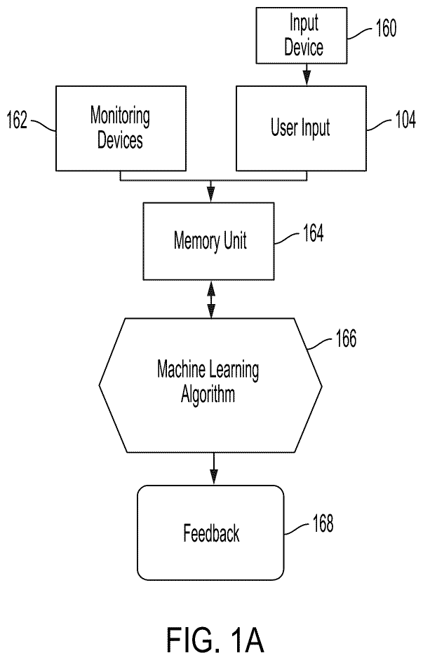

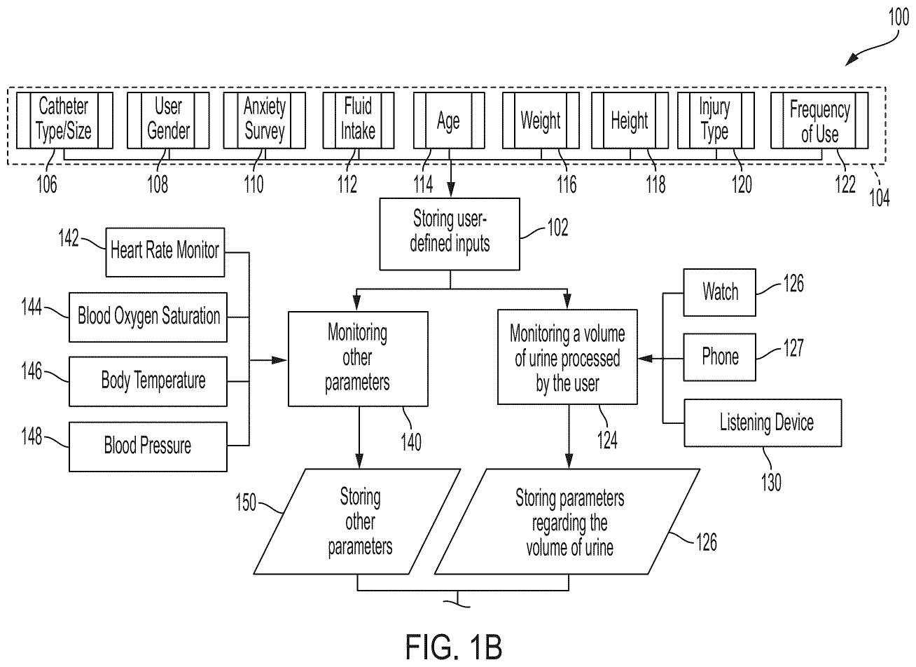

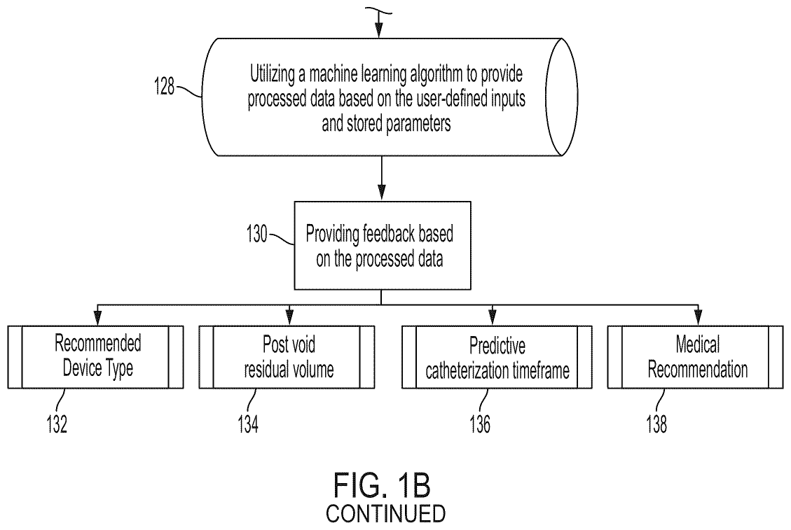

Referring to FIG. 1a, several components utilized to implement a urological health diagnostic system 100 are illustrated. More specifically, the diagnostic system 100 may utilize one or more input device 160. The input device 160 may be used by a user to input data into the diagnostic system 100 that can be stored therein as a user input 104. The input device 160 may be a remote device such as a computer, smartphone, tablet, smartwatch, or any other device capable of providing a user interface wherein the user can enter data to be stored in a memory unit 164. The term “smartphone” refers to any device capable of communicating with other devices and intended to be kept with an individual. Similarly, the term “smartwatch” refers to any wrist-worn device that is capable of communicating wirelessly with other devices.

The memory unit 164 may be any type of memory unit capable of storing and providing data. In one non-exclusive example, the memory unit 164 is a memory unit from the input device 160 such as solid state memory on a smartphone. Alternatively, the memory unit 164 may utilize a cloud-based protocol to manage and store data.

The diagnostic system 100 may have access to one or more monitoring device 162 as well. The monitoring device 162 may be any type of sensor capable of identifying a state of a user. For example, the monitoring device 162 may be a known sensor capable of identifying a user's heart rate. The monitoring device 162 may also include a known sensor capable of identifying a user's blood oxygen saturation, body temperature, and blood pressure. The monitoring devices 162 contemplated herein are generally known in the art. In one example of this disclosure, one or more of the monitoring devices 162 may be part of the input device 160 as well. For example, the input device may be a smartwatch that has one or more of a heart rate monitor, a blood oxygen saturation sensor, a body temperature sensor, and a blood pressure sensor. Further, the monitoring devices 162 considered herein may include the microphone, camera, and location services typically available through common input devices 160 such as tablets, smartphones, and smartwatches.

Data provided by the monitoring device 162 may be saved in the memory unit or passed directly to a machine-learning algorithm 166 being implemented by a processor. The machine learning algorithm 166 may be stored in the memory unit 164 or otherwise and configured to be executed by one or more processors commonly known in the art. For example, the processor of the input device 160 may implement some, or all, of the machine learning algorithm 166. Alternatively, a known cloud-based system may store and implement the machine learning algorithm 166. Regardless, the machine learning algorithm may have access to the data provided by the input device 160 and the monitoring devices 162.

The machine-learning algorithm may utilize any machine learning and/or artificial intelligence algorithm for performing the functions described herein. For example, in some embodiments, the machine-learning algorithm 166 may utilize one or more neural network algorithms, regression algorithms, instance-based algorithms, regularization algorithms, decision tree algorithms, Bayesian algorithms, clustering algorithms, association rule learning algorithms, deep learning algorithms, dimensionality reduction algorithms, and/or other suitable machine learning algorithms, techniques, and/or mechanisms.

The machine learning algorithm 166 may also have a feedback device 168 for providing feedback to a user. The feedback device 168 may be a screen for providing visual feedback to a user. The screen may be part of the input device 160 such as the screen of a smartphone, smartwatch, tablet, or the like. Alternatively, the screen may be entirely independent of the input device 160. The feedback device 168 may include an audio feedback through a speaker or the like. The speaker may be part of the input device 160 such as the speaker of a smartphone, smartwatch, tablet, or the like. Alternatively, the speaker may be entirely independent of the input device 160. The feedback device 168 may include haptic feedback through a vibrator or the like. The haptic feedback may be provided from the input device 160 such as the vibrator of a smartphone, smartwatch, tablet, or the like. Alternatively, the haptic feedback may be generated entirely independently of the input device 160. Any one or more combinations of the feedback devices discussed herein are considered part of this disclosure.

Referring now to FIG. 1b, a schematic view of the logic flow of the urological health diagnostic system 100 is illustrated. In one aspect of this disclosure, data for the health diagnostic system 100 may be stored in the memory unit 164 discussed herein. For example, the data may be stored locally, on remote servers, or utilize commonly known cloud-based protocols.

The teachings considered herein may be implemented by known hardware components. User inputs 104 may be input through the input devices 160 discussed herein among other ways. For example, a smartphone, smartwatch, computer, tablet, or the like may provide user input options for a user. Further, the user input may be transmitted to the memory unit 164 or database through known wired or wireless protocols. The memory unit 164 may then store the user input as data or use the information provided by the user input 104 to execute the diagnostic system 100. The diagnostic system 100 may be implemented using one or more processor from one or more input device 160 or may have a dedicated processor to implement the teachings discussed herein. Unless specifically stated otherwise, the one or more processor implementing the teachings discussed herein may be known hardware components that are understood by a person having ordinary skill in the art.

In an initial step of the diagnostic system 100, user inputs 104 may be stored as indicated in box 102. The user inputs 104 may be provided from any source, including the input devices 160 discussed herein among others. The user-defined inputs from box 102 can include one or more user input 104. The user input 104 may be a catheter type and/or size 106. For example, the user input 104 input and stored in box 102 may include the specific brand of catheter being used by a user or the gauge of the catheter tube as illustrated in box 106. Alternatively, or additionally, the user input 104 may include one or more of a user's gender 108, anxiety survey data 110, fluid consumption or intake 112, age 114, weight 116, height 118, injury type 120, frequency of use 122, activity level, diet survey data, blood pressure, skin color, skin turgor, extremities temperature, and respiratory rate among other things.

In one aspect of this disclosure, the temperature, respiratory rate, and blood pressure can be measured by the smartwatch. For example the blood pressure may be measured using optical heart rate sensors in the smartwatch. Similarly, the temperature may also be measured by temperature sensors in the smartwatch.

Regarding the skin color, any device having a camera or similar sensor may be directed towards a user's skin to determine the characteristics thereof. In one non-limiting example, a device like the Nix Pro provided by Nix Sensor Ltd. may be used.

Regarding skin elasticity, one type of sensor can be used to measure the skin turgor. Examples of such sensors comprise the Elastimeter or the SkinFibroMeter provided by Delfin Technologies. However, any sensor capable of determining skin elasticity is contemplated herein.

The user inputs 104 may be any information that may be helpful in evaluating the urological health of the user. For example, the anxiety survey data 110 may be considered to determine the user's level of anxiety when the diagnostic system 100 was implemented. Further, the fluid intake 112 may provide data input by the user or other source for the diagnostic system 100 to identify the volume of fluid consumed by the user. Further still, the injury type 120 may provide the diagnostic system 100 with information regarding the type of injury sustained by the user to thereby assist with understanding the potential urological conditions of the user, including the likelihood of autonomic dysreflexia among other things. Autonomic dysreflexia is a medical problem that can happen when the spinal cord is injured in the upper back. Under certain conditions, autonomic dysreflexia can make the blood pressure dangerously high coupled with very low heart rate, which can lead to a stroke, seizure, or cardiac arrest. Autonomic dysreflexia happens when the autonomic nervous system overreacts to a noxious stimulus below the damaged spinal cord. In one example considered herein, a full bladder can trigger autonomic dysreflexia.

Similarly, the frequency of use input 122 may identify how frequently the user urinates, either independently or with assistance from a medical device such as a urinary catheter. Regardless, the user inputs 104 are data selectively input by a user or otherwise obtained.

The user inputs 104 may be stored in one or more memory unit 164 for further processing. As considered herein, the user inputs 104 may be stored on remote servers or the like. Alternatively, the user inputs 104 may not be substantially stored but rather be immediately used by the diagnostic system 100.

The diagnostic system 100 may also monitor a volume of urine processed by the user in box 124. For this step, the diagnostic system may utilize a microphone on a smartwatch 126, smartphone 127, or other listening device 130 to determine the volume of urine processed by the user during a urinary event wherein the user voids some or all of the contents of the user's bladder (hereinafter “void event”). More specifically, the microphone from one or more of the smartwatch 126, smartphone 127, and listening device 130 may be used to identify the sounds typically generated during a void event. The audio signals generated during the void event may be stored and processed by the diagnostic system 100 to determine the fluid volume of urine processed during the void event. The other listening device 130 may be any device having a microphone capable of detecting a void event. In one non-exclusive example, the other listening device may be a smart speaker commonly capable of identifying a sound and wirelessly communicating with other devices.

The duration of the void event may be determined based on the audio signals generated. The diagnostic system 100 may have stored in the user inputs 104 information that may be considered for determining the volume of urine processed during the void event. As one example, a user that utilizes a urinary catheter may provide the catheter characteristics as user inputs via box 104. The diagnostic system 100 may utilize the machine learning algorithm 166 to establish an estimated volume of urine released during the void event using all or a subset of data provided by 104, 160, and 162. As one example, a user that utilizes a urinary catheter may provide the catheter type and size 106 as a user input 104. The diagnostic system 100 may have stored therein the expected flow rate of urine through a catheter having the user-input size. With this information, the diagnostic system 100 may utilize the machine learning algorithm 166 along with the expected flow rate for the specific catheter size and the flow duration to establish an estimated volume of urine released during the void event.

The diagnostic system 100 may store the parameter regarding the volume of urine produced during the void event in a database for later processing or consideration in box 126. The parameters regarding the volume of urine from box 126 may be further processed through the machine learning algorithm 166 in box 128. The machine learning algorithm 166 may be stored and implemented on one or more of the devices considered herein. More specifically, the machine learning algorithm 166 may be locally stored and executed on any one or more of the user's watch 126, phone 127, or any other personal computing device of the user. Additionally, some or all of the machine learning algorithm 166 may be stored on a remote server or cloud computing system. Regardless, the machine learning algorithm 166 processes the data provided thereto-which includes the user inputs 104 stored in box 102 and the parameters regarding the volume of urine from box 126.

In one aspect of this disclosure, the machine learning algorithm 166 is provided the basic parameters regarding the volume of urine from box 126. The basic parameters may only include the duration of an audio signal identified during the void event. From that information, along with the user inputs 104 from box 102, the machine learning algorithm 166 may determine the estimated volume of urine processed during the void event.

The machine learning algorithm 166 may also provide an estimate of insensible fluid loss using the information stored in the memory unit 164 that may include all or a subset of the user input 104, and a subset of the data from the monitoring device 162 such as the time history of heart rate variation, blood oxygen saturation, body temperature, and blood pressure among other things. Insensible fluid loss refers to the amount of body fluid lost daily that is not easily measured, from the respiratory system, skin, and water in the excreted stool. In part of this example, the feedback can include a predictive catheterization timeframe. The estimated insensible fluid loss will be used to determine the post void residual volume in 134. This may be an explicit estimation of the insensible fluid loss. Alternatively, the insensible fluid loss can be implicitly taken into account. In doing so, the machine learning algorithm 166 uses the above-mentioned data along with the estimated catheterized urine volume to improve the estimation accuracy of the post void residual volume, without providing an explicit number for the insensible fluid loss.

The machine learning algorithm implemented in box 128 may consider all of the information obtained and stored from boxes 102, 124, and 126 to provide processed data that is used to establish feedback on the feedback device 168 in box 130. The machine learning algorithm may determine feedback that may be any information about the user that may be beneficial in view of the processed data. More specifically, the machine learning algorithm 166 may consider the processed data to provide feedback such as a recommended device type 132 in instances where the user input 104 indicated that the user used a urinary catheter for the void event.

Further, the machine learning algorithm 166 may provide feedback regarding the expected residual volume of urine after the void event 134. This feedback may be generated by the machine learning algorithm 166 by considering user-inputs 104 regarding the fluid intake 112 of the user, the estimated insensible fluid loss, and the determined volume of urine processed by the user during the void event in boxes 124, 126, 128. The machine learning algorithm 166 may analyze trends regarding historical data for the user to generate expected volumes of urine to be output by the user during the void event. The expected volumes may be compared to the actual volume generated during the void event and the machine learning algorithm 166 may provide feedback showing any estimated post void residual volume of urine remaining in the user's bladder.

Identifying scenarios wherein the user has a residual volume of urine in the bladder post void event may further be considered by the machine learning algorithm 166 to provide feedback in the form of a medical recommendation 138. For example, if the machine learning algorithm identifies a variance from the user's typical post void residual volume, the feedback presented may indicate that the user should seek medical attention to resolve the inconsistent urological function. In other words, the machine learning algorithm 166 may maintain and access historical processed data to identify expected trends and the like. The historical processed data may be compared to any new processed data to identify any anomalies wherein the new processed data is not presenting the expected outcomes. This scenario may be indicative of a health condition, such a urinary tract infection, and the machine learning algorithm may alert the user of the issue via the feedback 130, 138.

The machine learning algorithm 166 may also generate feedback in the form of a predictive catheterization timeframe 136. More specifically, the machine learning algorithm 166 may assess historical data from the user or other general data regarding estimated bladder volumes based on the fluid intake 112 identified via the user input 104. In one example, the fluid intake 112 may include a timestamp and the machine learning algorithm 166 may consider both the volume of fluid consumed, the time the fluid was consumed, and historical data to generate an estimated volume of urine in the bladder. This estimated volume may be used to predict when the volume of fluid in the bladder is at a level where it should be voided to avoid damaging the urinary system. Accordingly, in one aspect of this disclosure the machine learning system 166 monitors the fluid intake 112, user input 104, and the data provided by the monitoring device 162 to provide feedback to the user predicting when the bladder should be voided via catheterization 136.

The machine learning algorithm may also consider and monitor other parameters in box 140. The other parameters may include data provided from sensors that can be used to identify heart rate 142, blood oxygen saturation 144, body temperature 146, and blood pressure 148 among others. Regardless of the type of sensor utilized, the other parameters from box 140 may be stored in box 150 at a location wherein the machine learning algorithm 166 has access to the data provided by the other parameters for consideration when providing the processed data and feedback of box 128. For example, one or more of the user's heart rate, blood oxygen saturation, body temperature, and blood pressure provided by the other parameters in box 140 may be compared to historical user data or a stored database to determine when the data provided by the other parameters may require medical attention, this may also include prediction of hydration status and risk of autonomic dysreflexia. More specifically, if the machine learning algorithm 166 determines that the user's heart rate is abnormally high or low, blood oxygen saturation is abnormally low, body temperature is abnormally high or low, or blood pressure is abnormally high or low it may provide feedback based on the processed data in 130 providing a medical recommendation 138 encouraging the user to seek medical attention to resolve the unexpected data from the other parameters 140.

The machine learning algorithm 166 may process data provided thereto continuously and does not require inputs from all of the sources considered herein before processing the data in box 128. For example, the machine learning algorithm 166 may immediately process data regarding fluid intake 112 provided via user input 104 regardless of whether a void event occurred. Similarly, the machine learning algorithm may continuously, iteratively, or selectively execute one or more of the monitoring boxes 124, 140. Accordingly, this disclosure contemplates utilizing the methods discussed herein in many different logic flows and those presented in FIG. 1b are examples of only one embodiment of the diagnostic system of this disclosure.

In use, a user may utilize their smartphone to provide inputs to the diagnostic system 100. For example, the user may open an application on their smartphone or the like prompting them to provide information regarding any one or more of the user inputs 104. The application may store the user data and prompt the user to initiate an audio recording during a void event. Alternatively, the user may engage an input device 160 such as a smart-assistant, smart watch, phone, or tablet to record the void event. Regardless, the recording of the void event and user inputs may be stored and accessed by the machine learning algorithm 166 to determine the volume of urine voided during the void event. The volume of urine voided may then be stored and processed by the application via the machine learning algorithm 166 and the user may be notified via the application if there is any noteworthy feedback identified by the machine learning algorithm 166.

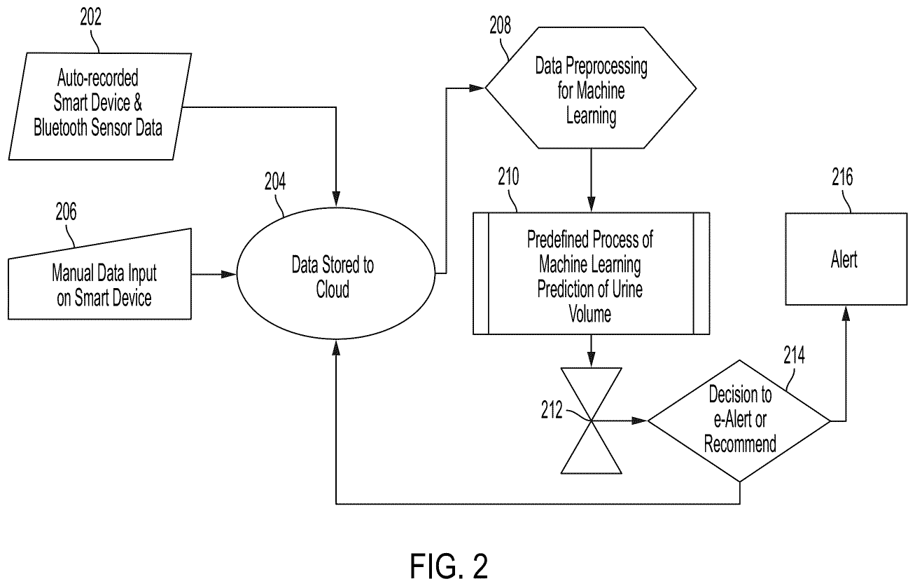

Referring now to FIG. 2, yet another embodiment of the present disclosure is illustrated. In this embodiment, sensor data is automatically recorded in box 202. The sensor data 202 is transmitted via known communication protocols, such as through internet transmission, to be stored on a cloud computing system in box 204. Further, box 206 provides for manual data input from a smart device. The manual data input of box 206 may include surveys and sound recordings of urination among other things. The manual data of box 206 may also be transmitted via known communication protocols, such as through internet transmission, to be stored on the cloud computing system of box 204.

The stored data of box 204 may be preprocessed for machine learning in box 208. In box 210, a predefined process of machine learning is used to predict the urine volume generated during the sound recording from box 206. In box 212, the machine learning algorithm analyzes the fluid intake and output, heart rate, anxiety level, and duration of catheterizations to determine whether an alert is recommended in box 214. If an alert is recommended, the alert may be presented in box 216 to one or more of the patient, clinicians, and caretakers. The alert may be presented via a smartwatch, smartphone, or other electronic device. Further, the data processed in box 212 may be reprocessed and/or stored as historical data to be further considered by the machine learning algorithm for future use.

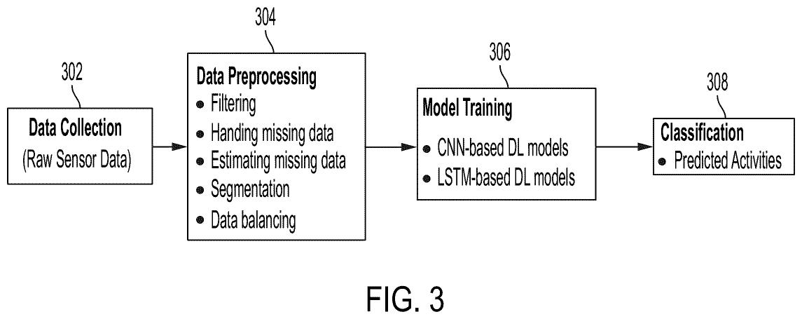

FIG. 3 shows one example of a data processing system contemplated herein. The data processing system may utilize data collection in box 302 that comprises the raw sensor data among other things. Data preprocessing may be executing in box 304. The data preprocessing may include filtering, handing missing data, estimating missing data, segmentation, and data balancing among other things. Box 306 may comprising model training such as CNN-based deep learning models and long short-term memory-based deep learning models among others. Finally, in box 308 a classification can occur wherein predicted activities are identified.

While this disclosure has been described with respect to at least one embodiment, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A method for identifying urological health information, comprising:
storing user-defined inputs provided by a user;
monitoring a fluid volume of urine processed by the user;
storing parameters regarding the fluid volume of urine;
utilizing a machine learning algorithm to provide processed data based on the user-defined inputs and stored parameters; and
providing feedback based on the processed data.

2. The method of claim 1, wherein the monitoring step comprises monitoring the volume of urine processed by a user through a urinary catheter.

3. The method of claim 2, further comprising using a microphone to record audio during a catheterization process to determine the fluid volume of urine transferred during the catheterization process with the machine learning algorithm.

4. The method of claim 3, further comprising storing details about the urinary catheter and considering the details to determine the fluid volume of urine transferred during the catheterization process.

5. The method of claim 4, further wherein the details comprise a urinary catheter gauge.

6. The method of claim 4, further wherein the details comprise the gender for which the urinary catheter is intended to be used.

7. The method of claim 3, further wherein the microphone is on a wristwatch.

8. The method of claim 3, further wherein the microphone is on a phone.

9. The method of claim 1, wherein the user-defined inputs comprises a survey identifying anxiety.

10. The method of claim 4, wherein the user-defined inputs comprises fluid intake volume.

11. The method of claim 10, further comprising determining a post-void residual volume with the machine learning algorithm based on the fluid intake volume, the fluid volume of urine transferred during the catheterization process, and data provided by the user and one or more monitoring device.

12. The method of claim 10, wherein feedback includes a predictive catheterization timeframe.

13. The method of claim 1, wherein the user-defined inputs comprises a device type.

14. The method of claim 1, wherein the user-defined inputs comprise one or more of gender, age, weight, height, specific injury, frequency of device use, and survey data.

15. The method of claim 1, further comprising gathering and storing heart rate data and considering the heart rate data with the machine learning algorithm before providing feedback.

16. The method of claim 1, wherein the feedback comprises a recommendation for device use frequency.

17. The method of claim 1, wherein the feedback comprises a recommendation for device type.

18. The method of claim 1, wherein the feedback comprises a medical recommendation.

19. The method of claim 1, further comprising identifying and considering one or more of blood oxygen saturation, blood pressure, heart-rate variability, body temperature, and heart rate with the machine learning algorithm.

20. The method of claim 1, further comprising storing sensor measurements providing a state of the user, wherein the sensor measurements include one or more of blood pressure, heart rate, and blood oxygen saturation.
